(19) United States
(12) Patent Application Publication
(10) Pub. No.: US 2024/0307664 A1
(43) Pub. Date: Sep. 19, 2024

(54) ASSEMBLIES AND METHODS FOR INTRODUCING AN ADDITIONAL GUIDEWIRE

(71) Applicant: BLUE WIRE LTD, Haifa (IL)

(72) Inventors: Elias HELLOU, Haifa (IL); Omri NAVEH, Alon Hagalil (IL)

(21) Appl. No.: 18/277,875

(22) PCT Filed: Feb. 22, 2022
(86) PCT No.: PCT/IB2022/051535

§ 371 Date: Aug. 18, 2023

Related U.S. Application Data
(60) Provisional application No. 63/151,825, filed on Feb. 22, 2021.

(51) Int. Cl. A61M 25/09

(52) U.S. Cl. CPC A61M 25/09041; A61M 2025/09108; A61M 2025/09125

(57) ABSTRACT

Embodiments of the present invention relate generally to medical devices and more particularly to methods and apparatus for introducing a second guidewire to a previously-introduced guidewire in a lumen of a subject. Assemblies, systems, apparatus, methods and kits (for example, for introducing an additional guidewire), provided in accordance with embodiments of the invention, are disclosed herein.

### FIELD OF THE INVENTION

Embodiments of the present invention relate generally to medical devices and more particularly to methods and apparatus for introducing a second guidewire to a previously-introduced guidewire in a lumen of a subject.

### BACKGROUND

Guidewires are used for navigating to reach a location within a patient such as a surgical or other treatment site. Once the tip of the guidewire arrives at its destination, it acts as a guide that larger elements, e.g., catheters, can rapidly follow for easier delivery to the treatment site. Introduction of a second guidewire in parallel to a previously-introduced guidewire can be useful in many different procedures. In order to ensure that the second guidewire follows the same path as the first, the introduction can be done using a coupling device.

‘Coupling tips’ designed to be affixed to the distal end of the second guidewire have been suggested, but to date all such coupling tips have suffered from a number of design flaws. The most serious flaws include: the pathway provided for the previously-introduced guidewire through the coupling tip is not uniformly parallel to the second guidewire, or close enough to parallel; the pathway provided for the previously-introduced guidewire through the coupling tip is not at a fixed orientation to the second guidewire, i.e., the orientation can change when an external force is applied on the coupling device; the pathway provided for the previously-introduced guidewire through the coupling tip is not uniformly straight, i.e., the previously-introduced guidewire is forced to bend, often in more than one place, as the coupling device traverses the length of the guidewire; the pathway provided for the previously-introduced guidewire through the coupling tip is too short; and the passage provided in the coupling tip for the distal end of the second guidewire is not adaptable to the diameter of the guidewire. Any or all of these design flaws can mean that when introducing the second guidewire using such coupling tips, the surgeon can meet resistance and inadvertently cause shear forces on the previously-introduced guidewire, or even force uncouple the coupling device from the second guidewire. Therefore, there is a need for assemblies and methods employing a coupling tip design that reduces or eliminates the foregoing disadvantageous effects.

### SUMMARY OF THE INVENTION

According to embodiments disclosed herein, an apparatus comprises: (a) a first guidewire for use in a medical procedure; and (b) a coupling device comprising: (i) a first tubular portion formed to accommodate, through at least a proximal majority of a length thereof, passage of a distal portion of the first guidewire, and (ii) a second tubular portion formed to accommodate slidable passage therethrough of a second guidewire in a straight-line path between respective proximal and distal openings of the second tubular section, the straight-line path having a length-to-diameter ratio of at least 10:1, the first and second tubular sections being joined laterally in a fixed orientation to each other along the lengths of respective overlapping longitudinal sections, the lateral joining being such that respective longitudinal centerlines of the first and second tubular portions are parallel to each other or within ±10° of parallel in said fixed orientation.

In some embodiments, the lateral joining can be such that the respective longitudinal centerlines are parallel to each other or within ±5° of parallel, in said fixed orientation.

In some embodiments, the lateral joining can be such that a distance between respective distal ends of the first and second tubular sections is less than 2 cm. In some embodiments, the lateral joining can be such that a distance between respective distal ends of the first and second tubular sections is less than 1 cm. In some embodiments, the lateral joining can be such that a distance between respective distal ends of the first and second tubular sections is less than 0.5 cm.

In some embodiments, the respective overlapping longitudinal sections can have a length of at least 1 cm. In some embodiments, the respective overlapping longitudinal sections can have a length of at least 2 cm. In some embodiments, the respective overlapping longitudinal sections can have a length of at least 3 cm. In some embodiments, the respective overlapping longitudinal sections can have a length of at least 4 cm.

In some embodiments, the length of the second tubular portion can be at least 1 cm. In some embodiments, the length of the second tubular portion can be at least 2 cm. In some embodiments, the length of the second tubular portion can be at least 3 cm. In some embodiments, the length of the second tubular portion can be at least 4 cm.

In some embodiments, the apparatus can be in an assembled state in which the coupling device is affixed to the distal portion of the first guidewire such that a longitudinal section of the first tubular portion surrounds the distal portion of the first guidewire. In some such embodiments, at least part of the longitudinal section of the first tubular portion that surrounds the distal portion of the first guidewire can be mechanically and/or thermally deformed as a result of an affixing process.

In some embodiments, the apparatus can additionally comprise an assembly material used in an affixing process.

According to embodiments disclosed herein, an apparatus provided as a kit comprises: (a) a coupling device for use in a medical procedure, the coupling device comprising: (i) a first tubular portion formed to accommodate, through at least a proximal majority of a length thereof, passage of a distal portion of a first guidewire, and (ii) a second tubular portion formed to accommodate slidable passage therethrough of a second guidewire in a straight-line path between respective proximal and distal openings of the second tubular section, the straight-line path having a length-to-diameter ratio of at least 10:1, the first and second tubular sections being joined laterally in a fixed orientation to each other along the lengths of respective overlapping longitudinal sections such that respective longitudinal centerlines of the first and second tubular portions are parallel to each other or within ±10° of parallel, or parallel to each other or within ±5° of parallel. The apparatus additionally comprises (b) an assembly aid for affixing the coupling device to a distal portion of the first guidewire, the assembly aid selected from the group of assembly aids consisting of assembly materials and assembly tools.

In some embodiments, the respective overlapping longitudinal sections can be parallel to each other or within ±5° of parallel.

In some embodiments, the apparatus can additionally comprise the first guidewire.

In some embodiments, the apparatus can be in an assembled state in which the coupling device is affixed to the distal portion of the first guidewire such that a longitudinal section of the first tubular portion surrounds the distal portion of the first guidewire. In some such embodiments, the assembly aid can comprise an assembly material, and the apparatus in the assembled state can comprise at least a portion of the assembly material.

In some embodiments, the assembly aid can comprise an assembly tool, and at least part of the longitudinal section of the first tubular portion that surrounds the distal portion of the first guidewire can be mechanically and/or thermally deformed by use of the assembly tool in an affixing process.

A method is disclosed, according to embodiments, for coupling a first guidewire to a previously introduced second guidewire during a medical procedure. The method comprises: (a) affixing a coupling device to a distal portion of the first guidewire, the coupling device comprising two first tubular portions joined laterally in a fixed orientation to each other along the lengths of respective overlapping longitudinal sections, the lateral joining being such that respective longitudinal centerlines of the first and second tubular portions are parallel to each other or within ±10° of parallel in said fixed orientation, the affixing being such that that a longitudinal section of a first tubular portion of the coupling device surrounds a distal portion of the first guidewire; and (b) slidably passing an end of the second guidewire through a second tubular portion of the coupling device in a straight-line path between respective proximal and distal openings of the second tubular section, the straight-line path having a length-to-diameter ratio of at least 10:1.

In some embodiments, the respective longitudinal centerlines can be parallel to each other or within ±5° of parallel in said fixed orientation.

In some embodiments, the lateral joining can be such that a distance between respective distal ends of the first and second tubular sections is less than 2 cm. In some embodiments, the lateral joining can be such that a distance between respective distal ends of the first and second tubular sections is less than 1 cm. In some embodiments, the lateral joining can be such that a distance between respective distal ends of the first and second tubular sections is less than 0.5 cm.

In some embodiments, the respective overlapping longitudinal sections of each of the first and second tubular portions can have a length of at least 1 cm. In some embodiments, the respective overlapping longitudinal sections of each of the first and second tubular portions can have a length of at least 2 cm. In some embodiments, the respective overlapping longitudinal sections of each of the first and second tubular portions can have a length of at least 3 cm. In some embodiments, the respective overlapping longitudinal sections of each of the first and second tubular portions can have a length of at least 4 cm.

In some embodiments, the length of the second tubular portion can be at least 1 cm. In some embodiments, the length of the second tubular portion can be at least 2 cm. In some embodiments, the length of the second tubular portion can be at least 3 cm. In some embodiments, the length of the second tubular portion can be at least 4 cm.

In some embodiments, a maximum internal diameter of the second tubular portion can be not greater than 2 mm. In some embodiments, a maximum internal diameter of the second tubular portion can be not greater than 1 mm.

In some embodiments, the affixing can include mechanically and/or thermally deforming at least part of the longitudinal section of the first tubular portion that surrounds the distal portion of the first guidewire using an assembly tool.

In some embodiments, the affixing can include using an assembly material.

A method is disclosed, according to embodiments, for coupling a first guidewire to a previously introduced second guidewire during a medical procedure. The method comprises: (a) providing an assembly comprising a first guidewire for use in a medical procedure, and a coupling device comprising first and second tubular portions joined laterally in a fixed orientation to each other along the lengths of respective overlapping longitudinal sections, the lateral joining being such that respective longitudinal centerlines of the first and second tubular portions are parallel to each other or within ±10° of parallel in said fixed orientation, the coupling device being affixed to the distal portion of the first guidewire such that a longitudinal section of the first tubular portion surrounds the distal portion of the first guidewire, and (b) slidably passing an end of the second guidewire through a second tubular portion of the coupling device in a straight-line path between respective proximal and distal openings of the second tubular section, the straight-line path having a length-to-diameter ratio of at least 10:1.

In some embodiments, the respective longitudinal centerlines can be parallel to each other or within ±5° of parallel in said fixed orientation.

In some embodiments, the lateral joining can be such that a distance between respective distal ends of the first and second tubular sections is less than 1 cm. In some embodiments, the lateral joining can be such that a distance between respective distal ends of the first and second tubular sections is less than 2 cm. In some embodiments, the lateral joining can be such that a distance between respective distal ends of the first and second tubular sections is less than 0.5 cm.

In some embodiments, the respective overlapping longitudinal sections of each of the first and second tubular portions can have a length of at least 1 cm. In some embodiments, the respective overlapping longitudinal sections of each of the first and second tubular portions can have a length of at least 2 cm. In some embodiments, the respective overlapping longitudinal sections of each of the first and second tubular portions can have a length of at least 3 cm. In some embodiments, the respective overlapping longitudinal sections of each of the first and second tubular portions can have a length of at least 4 cm.

In some embodiments, the length of the second tubular portion can be at least 1 cm. In some embodiments, the length of the second tubular portion can be at least 2 cm. In some embodiments, the length of the second tubular portion can be at least 3 cm. In some embodiments, the length of the second tubular portion can be at least 4 cm.

In some embodiments, a maximum internal diameter of the second tubular portion can be not greater than 2 mm. In some embodiments, a maximum internal diameter of the second tubular portion can be not greater than 1 mm.

In some embodiments, at least part of the longitudinal section of the first tubular portion that surrounds the distal portion of the first guidewire can be mechanically and/or thermally deformed as a result of an affixing process.

In some embodiments, the apparatus can additionally comprise an assembly material used in an affixing process.

A method is disclosed, according to embodiments, for assembling an apparatus. The method comprises: (a) providing a guidewire; (b) providing a coupling device comprising a first tubular portion, and a second tubular portion joined laterally in a fixed orientation to the first tubular portion along the lengths of respective overlapping longitudinal sections; and (c) affixing the coupling device to the distal portion of the guidewire, such that that a longitudinal section of a first tubular portion of the coupling device surrounds a distal portion of the guidewire, wherein the second tubular portion is formed to accommodate slidable passage therethrough of a different guidewire in a straight-line path between respective proximal and distal openings of the second tubular section, the straight-line path having a length-to-diameter ratio of at least 10:1.

In some embodiments, the affixing can include mechanically and/or thermally deforming at least part of the longitudinal section of the first tubular portion that surrounds the distal portion of the first guidewire using an assembly tool.

In some embodiments, the affixing can include using an assembly material.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1A shows a schematic side view of a coupling device according to embodiments of the present invention.

FIG. 1B shows, schematically, various non-limiting examples of cross-sections of coupling devices, including the coupling device of FIG. 1A, according to embodiments of the present invention.

FIG. 2A shows a schematic side view of an apparatus comprising a guidewire and a coupling device according to embodiments of the present invention.

FIG. 2B shows the apparatus of FIG. 2A in perspective view.

FIGS. 3 and 4 show schematic side views of an apparatus comprising a guidewire and a coupling device, in an assembled state, coupled to the proximal end of another guidewire, according to embodiments of the present invention.

FIGS. 5A and 5B show schematic side views of an apparatus comprising a guidewire and a coupling device, in an assembled state, coupled to the proximal end of another guidewire, and an angle between respective longitudinal centerlines of the two tubular portions of the coupling device, according to embodiments of the present invention.

FIG. 6 shows a schematic side view of an apparatus comprising a guidewire and a coupling device, in an assembled state, coupled to the proximal end of another guidewire, and an angle between respective longitudinal centerlines of the two guidewires, according to embodiments of the present invention.

FIGS. 7A and 7B show schematic side views of an apparatus comprising a guidewire and a coupling device, in an assembled state, coupled to the proximal end of another guidewire, and a distance between respective openings of the two tubular portions of the coupling device, according to embodiments of the present invention.

FIG. 7C shows a schematic side view of an apparatus comprising a guidewire and a coupling device, in an assembled state, coupled to the proximal end of another guidewire, wherein at least one of the two tubular portions is not continuous, according to embodiments of the present invention.

FIG. 8 shows a schematic side view of an apparatus comprising a guidewire and a coupling device, in an assembled state, coupled to the proximal end of another guidewire, the tubular portions having beveled distal and proximal openings, according to embodiments of the present invention.

FIG. 9 schematically illustrates a step of a method for assembling an apparatus, according to embodiments of the present invention.

FIGS. 10A and 10B schematically illustrate steps of respective methods for assembling an apparatus using an assembly aid, according to embodiments of the present invention.

FIGS. 11A, 11B and 11C are schematic end-view drawings of respective non-limiting examples of an apparatus comprising a guidewire and a coupling device, in an assembled state, according to embodiments of the present invention.

FIGS. 12A, 12B, 12C, 13A, 13B, 14A and 14B are schematic illustrations of apparatuses provided as kits, according to embodiments of the present invention.

FIGS. 15A, 15B, 15C and 15D illustrate steps of a method for coupling a first guidewire to a previously introduced second guidewire during a medical procedure, according to embodiments of the present invention.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

The terms ‘distal’ and ‘proximal’ are used throughout this disclosure and the appended claims as follows: ‘distal’ means further into the body (along an insertion path) from a point of entry into the body, while ‘proximal’ means closer to the point of entry into the body. Where the terms are used in reference to apparatus outside of a patient's body, a distal portion or distal end is that portion or end of the apparatus configured to be inserted into the body first, while a proximal end or proximal portion is either inserted last or may never be inserted (as in the case of a guidewire, for example). Additionally, when used relatively, e.g. ‘distally displaced from’ or ‘proximal of’, the meaning is, respectively, closer to the distal end than’ or ‘closer to the proximal end than’. The respective distal and proximal directions are shown in many of the figures, and in all relevant figures the convention of ‘distal to the left’ and ‘proximal to the right’ is maintained.

The terms “first” and “second” with respect to guidewires and respective portions of coupling devices are used throughout this disclosure and in the appended claims in accordance with the following convention: The “first guidewire” means the guidewire that a surgeon affixes to a coupling device according to embodiments, and then couples to a guidewire previously introduced in a lumen of a patient. Thus, the previously-introduced guidewire is referred to as the “second guidewire”. Similarly, a ‘first’ tubular portion of the coupling device is for accommodating therein a portion of the first guidewire, and the ‘second’ tubular portion of the coupling device is for accommodating the passage therethrough of the second guidewire.

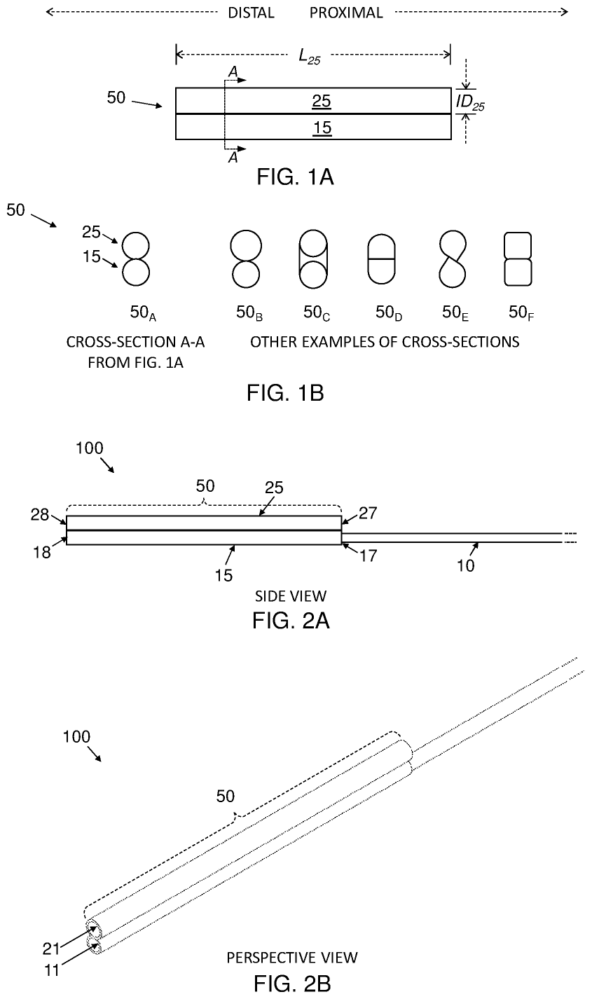

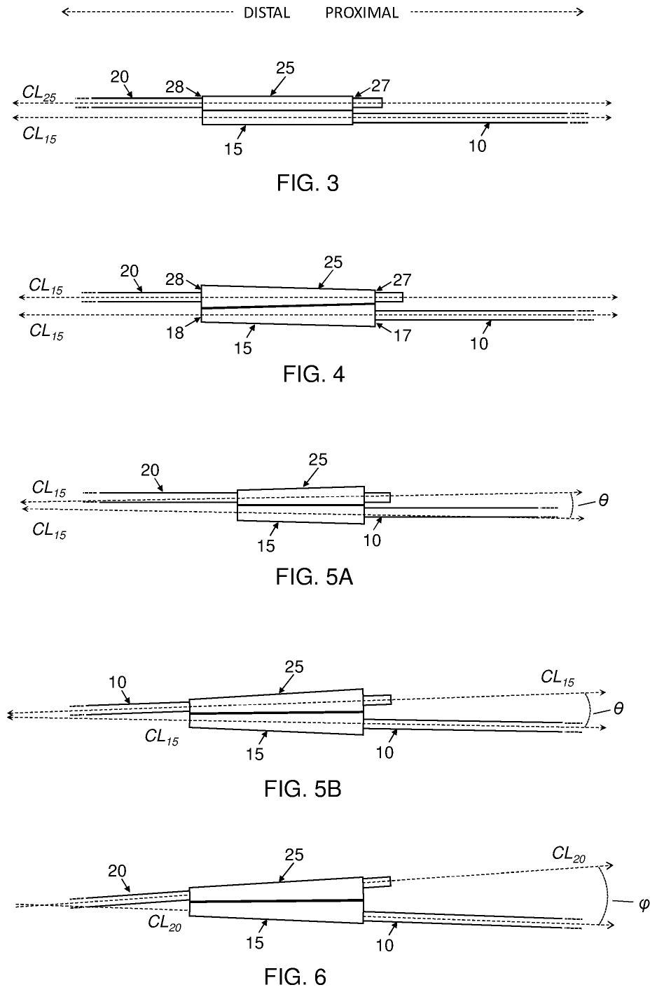

According to embodiments, a coupling device is provided for coupling a first guidewire to a second guidewire that has already been introduced to a lumen. A non-limiting example of a coupling device 50 is illustrated schematically in FIG. 1A. The coupling device of FIG. 1A includes first tubular section 15 for accommodating, through at least a proximal majority of a length of the first tubular section 15, passage of a distal portion of the first guidewire 10 (e.g., any of the first guidewires 10 of FIGS. 2A-15D), and a second tubular section 10 for accommodating therethrough slidable passage of a second guidewire 20 (e.g., any of the second guidewires 20 of FIG. 3-8 or 15A-15D). In embodiments, a coupling device is formed from a flexible or elastic material, such as a polymer or a metal (or metal alloy), or from a super-elastic material, such as a nitinol.

As discussed hereinabove, when the portion of the coupling device 50 meant to be slidable along the second guidewire 20 (the previously-introduced guidewire) is too short, it can be more difficult to slide the coupling device 50 without damaging the guidewire 20 or getting stuck. Accordingly, the second tubular section 25 is provided, according to embodiments, with both a minimum length and a minimum length-to-diameter ratio that in combination equip the coupling device 50 with an enhanced ability to traverse the second guidewire 20 safely and effectively. The second tubular section 25 has a length indicated in FIG. 1 by the arrow marked L₂₅, and an inner diameter marked ID₂₅. In embodiments, the length L₂₅ is suitably at least 1 cm. In some embodiments, the length L₂₅ is suitably at least 2 cm. In other embodiments, the length L₂₅ is suitably at least 3 cm. In yet other embodiments, the length L₂₅ is suitably at least 4 cm. In embodiments, the inner diameter ID₂₅ is suitably at least 0.95 mm and no more than 2 mm. In some embodiments, the inner diameter ID₂₅ is suitably at least 0.95 mm and no more than 3 mm. In some other embodiments, the inner diameter ID₂₅ is suitably at least 0.5 mm and no more than 2 mm. In some other embodiments, the inner diameter ID₂₅ is suitably at least 0.5 mm and no more than 1.5 mm. In yet other embodiments, the inner diameter ID₂₅ is suitably at least 0.5 mm and no more than 3 mm. In still other embodiments, the inner diameter ID₂₅ is suitably at least 0.2 mm and no more than 2 mm. In yet other embodiments, the inner diameter ID₂₅ is suitably at least 0.2 mm and no more than 1 mm. In still other embodiments, the inner diameter ID₂₅ is suitably at least 0.2 mm and no more than 3 mm. The ratio of the length Les to the inner diameter ID₂₅ is suitably between 100:1 and 5:1, or between 50:1 and 5:1, or between 30:1 and 5:1, or between 100:1 and 10:1, or between 50:1 and 10:1, or between 30:1 and 10:1, or between 100:1 and 30:1, or between 50:1 and 30:1, all ranges inclusive. In any of the embodiments, an inner diameter ID 15 of the first tubular portion 15 (not shown) can be the same as the inner diameter ID₂₅ of the first tubular portion 25, or within ±0.1 mm, or within ±0.2 mm, or within ±0.3 mm, or within ±0.4 mm, or within ±0.5 mm, or within ±0.6 mm, or within ±0.7 mm, or within ±0.8 mm, or within ±0.9 mm, or within ±1.0 mm, or within ±1.1 mm, or within ±1.25 mm, or within ±1.5 mm of the inner diameter ID₂₅ of the first tubular portion 25.

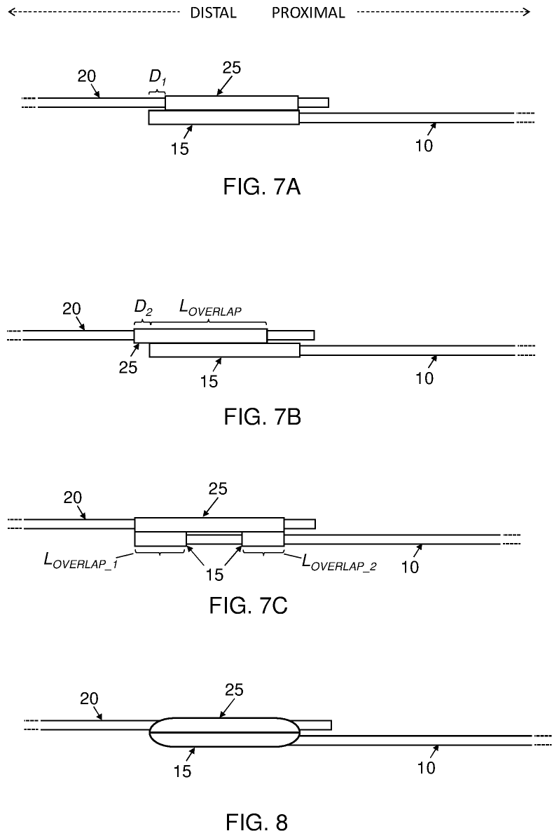

As illustrated in FIGS. 1A and 1B, the first and second tubular portions 15, 25 are joined laterally to each other along respective longitudinal sections. In some examples of coupling devices 50, the respective longitudinal may be less than fully overlapping, e.g., as illustrated in FIGS. 7A-7B, or may not be continuous sections (and/or at least one of the first and second tubular portions 15, 25 may not be continuous, as illustrated in FIG. 7C).

FIG. 1B shows exemplary cross-sectional views of a coupling device 50 according to various embodiments. In all of the examples, the lower-illustrated portion, as is shown in FIG. 1B for coupling device 50_{A}, is the first tubular portion 15 and the upper portion is the second tubular portion 25. Coupling device 50_{A} is the A-A cross-sectional view of the coupling device 50 of FIG. 1A, and indicates that the respective first and second tubular portions 15, 25 are cylinders (or, alternatively, conic sections) of equal diameters and laterally joined along respective tangential longitudinal sections in a fixed orientation to each other. In contrast, in coupling device 50_{B}, the second tubular portion 25 has a larger diameter than the first tubular portion 15. In other examples (not shown), the second tubular portion 25 can have a smaller diameter than the first tubular portion 15. Exemplary coupling device 50_{C} illustrates a lateral joining of the first and second tubular portions 15, 25 without the tubular portions 15, 25 actually touching each other. Exemplary coupling device 50_{D} illustrates a lateral joining of the first and second tubular portions 15, 25 in which the contact between the tubular portions 15, 25 is more than just tangential, in contrast to, e.g., coupling device 50_{A}. Exemplary coupling devices 50_{E} and 50_{F} are additional non-limiting examples of the broad range of possible cross-sections of coupling devices 50, any of which are within the scope of this invention.

Reference is made to FIGS. 2A and 2B. A distal portion of a first guidewire 10 resides within the lumen 11 of the first tubular portion 15 of a coupling device 50 according to embodiments. A distal portion of the first guidewire 10 enters the lumen 11 of the first tubular portion 15 through its proximal opening 17 but in the illustrated example does not exit the distal opening 18. In some examples the distal tip of the first guidewire 15 can extend slightly past the distal opening 18, e.g., by <1 mm, by <2 mm, by <3 mm, by <4 mm or by <5 mm. In some examples, the distal portion of the first guidewire 10 does not traverse the entire first tubular portion 15, but only a proximal majority of the length of the first tubular portion 15.

FIG. 3 illustrates a first use-case example of an apparatus 100, in an assembled state such that the distal portion of the first guidewire 10 passes through at least a proximal majority of the first tubular section 15. A second guidewire 20 passes through the lumen 21 of the second tubular section 25 in a straight path from the distal opening 28 to the proximal opening 27. In actual use, the second guidewire 20 is substantially stationary as it has already been introduced to the lumen of a patient, and it is the second tubular portion 25 of the coupling device 50 that actively slides along the length of the second guidewire 25. As the motion of the coupling device 50 is relative to the guidewire 20, one can say, equivalently, that the second guidewire 20 passes slidably through the second tubular portion 25. Respective longitudinal centerlines CL₁₅, CL₂₅ of the first and second tubular portions 15, 25 of the coupling device 50 are shown in FIG. 3. The two centerlines CL₁₅, CL₂₅ are parallel to each other, indicating that the first and second guidewires 10, 20 can be accommodated within the first and second tubular portions 15, 25 in parallel paths.

In a second use-case example illustrated in FIG. 4, the first and second tubular portions have a conic-frustum shape, reversed relative to each other, such that the distal opening 28 of the second tubular section 25 is larger than the distal opening 18 of the first tubular section 15, and the proximal opening 22 of the second tubular section 25 is smaller than the proximal opening 17 of the first tubular section 15. Nonetheless, the coupling device 50 of FIG. 4 has been arranged such that the two centerlines CL₁₅, CL₂₅ are parallel to each other, as was the case in the example illustrated in FIG. 3.

In a third use-case example illustrated in FIG. 5A, the first and second tubular portions 15, 25 have the same conic-frustum shape as the coupling device 50 of FIG. 4, but are not reversed as was the case in FIG. 4. The lateral joining of the two tubular portions 15, 25 causes them to be arranged such that the two centerlines CL₁₅, CL₂₅ are not parallel to each other. Instead, the two centerlines CL₁₅, CL₂₅ are rotated relative to each other to be oriented with an angle θ between them. In some embodiments, θ≤5° in either direction, i.e., θ≤+5° such that the two centerlines CL₁₅, CL₂₅, if not parallel, are within ±5° of being parallel.

In a fourth use-case example illustrated in FIG. 5B, the first and second tubular portions 15, 25 have a right-frustum shape, and are longer than the first and second tubular portions 15, 25 of FIG. 5A. The lateral joining of the two tubular portions 15, 25 along respective longitudinal sections causes them to be arranged such that the two centerlines CL₁₅, CL₂₅ are not parallel to each other. Instead, the two centerlines CL₁₅, CL₂₅ are rotated relative to each other to be oriented with an angle θ between them. In some embodiments, θ≤5° in either direction, i.e., θ≤+5° such that the two centerlines CL₁₅, CL₂₅, if not parallel, are within ±5° of being parallel. In some other embodiments, θ≤10° in either direction, i.e., θ≤+10° such that the two centerlines CL₁₅, CL₂₅, if not parallel, are within ±10° of being parallel.

In a fifth use-case example illustrated in FIG. 6, the freedom of the guidewires 10, 20 to pass through the first and second tubular portions 15, 25 is illustrated by showing a maximum rotation of the guidewires 10, 20 relative to each other to be oriented with an angle φ between the respective centerlines CL₁₀, CL₂₀ of the guidewires 10, 20. In some embodiments, φ≤5° in either direction, i.e., φ≤±5° such that the two centerlines CL₁₀, CL₂₀, if not parallel, are within ±5° of being parallel. In some other embodiments, φ≤10° in either direction, i.e., φ≤±10° such that the two centerlines CL₁₀, CL₂₀, if not parallel, are within ±10° of being parallel.

In each of the use-case examples illustrated in FIGS. 3-6, the first and second tubular portions 15, 25 of each example completely overlap with each other. In sixth and seventh use-case examples, illustrated, respectively, in FIGS. 7A and 7B, the first and second tubular portions 15, 25 do not completely overlap along their entire respective lengths. In FIG. 7A, the lateral joining of the first and second tubular portions 15, 25 is such that the distal end of the first tubular section 15 is displaced distally from the distal end of the second tubular section 25 by a distance D₁. In FIG. 7B, the lateral joining is such that the distal end of the second tubular section 25 is displaced distally from the distal end of the first tubular section 15 by a distance D₂. Non-zero values of D1 or D2 can be desirable, in some cases, to improve usability of the coupling device 50, although they are not required in order to practice the embodiments. In embodiments, both D₁ and D₂ are limited to being less than or equal to 2 cm. In some embodiments, both D₁ and D₂ are limited to being less than or equal to 1 cm. In other embodiments, both D₁ and D₂ are limited to being less than or equal to 0.5 cm. The longitudinal overlap of the first and second tubular portions 15, 25, for example the longitudinal overlap L_{OVERLAP} shown in FIG. 7B, can contribute to the usability and/or efficacy of the coupling device 50. In embodiments, longitudinal overlap L_{OVERLAP} is at least 1 cm in length. In some embodiments, longitudinal overlap L_{OVERLAP} is at least 2 cm in length. In other embodiments, longitudinal overlap L_{OVERLAP} is at least 3 cm in length. In still other embodiments, longitudinal overlap L_{OVERLAP} is at least 4 cm in length.

The longitudinal overlap between the laterally joined first and second tubular portions 15, 25 can be a sum of multiple overlapping longitudinal sections, as illustrated in an eighth use-case example illustrated in FIG. 7C. The first tubular portion 15 includes multiple sections which may or may not be connected to each other. Each section can form a corresponding longitudinal overlap L_{OVERLAP} with the second tubular portion 25, e.g., longitudinal overlap sections L_{OVERLAP_1} and L_{OVERLAP_2} of FIG. 7C. In embodiments, the sum of L_{OVERLAP_1} and L_{OVERLAP_2} is at least 1 cm. In some embodiments, the sum of L_{OVERLAP_1} and L_{OVERLAP_2} is at least 2 cm. In some embodiments, the sum of L_{OVERLAP}_I and L_{OVERLAP_2} is at least 3 cm. In other embodiments, the sum of L_{OVERLAP_1} and L_{OVERLAP_2} is at least 4 cm.

In a ninth use-case example illustrated in FIG. 8, the distal and/or proximal ends of the first and/or second tubular portions 15, 25 can have a curved or beveled profile to improve usability and efficacy. In other examples, the beveling can be only at the distal ends and/or can create smaller bevels and/or rounded edges of the distal ends.

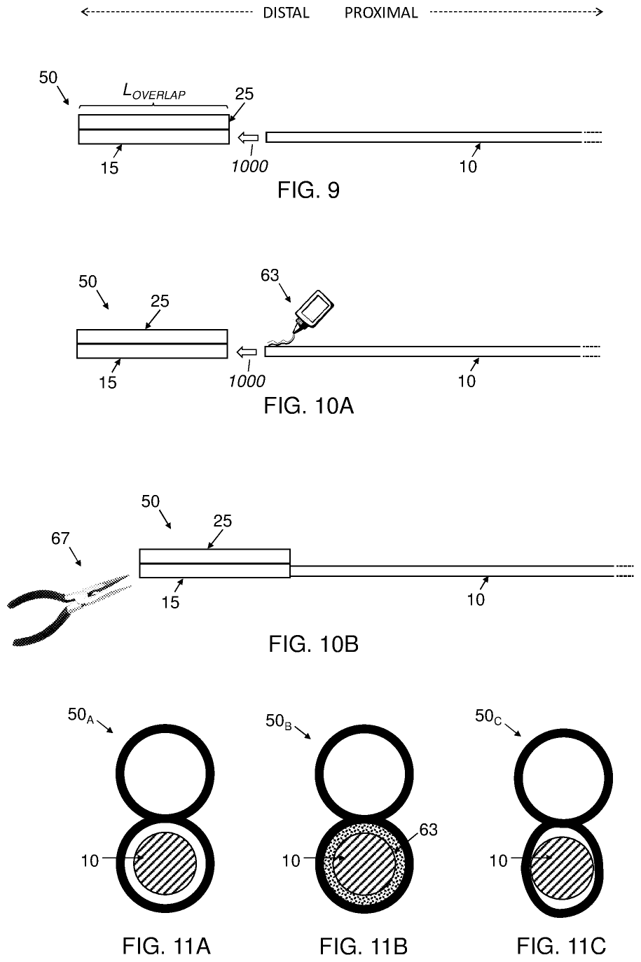

FIGS. 9, 10A and 10B illustrate some steps of a method for assembling an apparatus including a guidewire and a coupling device. FIG. 9 illustrates the method steps of (a) providing a first guidewire 10 and (b) providing a coupling device 50 comprising a first tubular portion 15, and a second tubular portion 25 joined laterally in a fixed orientation to the first tubular portion 15 along the lengths of respective overlapping longitudinal sections marked L_{OVERLAP} in FIG. 9. The coupling device 50 can be any of the coupling devices 50 disclosed herein, including coupling devices 50 discussed with respect to any one or more of FIGS. 1A-8. As indicated by arrow 1000 in FIGS. 9 and 10A, the method also includes the step of affixing the coupling device 50 to the distal portion of the guidewire 10 by inserting the distal end of the guidewire 10 into the first tubular portion 15 of the coupling device 50, such that such that a longitudinal section of the first tubular portion 15—at least a proximal majority of the first tubular section 15—surrounds a distal portion of the guidewire 10. FIG. 11A is a schematic end view showing the distal portion of the guidewire 10 surrounded by the first tubular portion 15.

In some embodiments, as illustrated schematically in FIG. 10A, the affixing step includes the use of an assembly aid, and specifically an assembly material 63. The assembly material 63 can include, for example, but not exhaustively: an adhesive; a mechanical element interposed between the distal portion of the guidewire 10 and the first tubular portion 15 such as, in a non-limiting example, a shim; and/or any other material effective to secure the guidewire 10 within the first tubular section 15, including, in non-limiting examples, by adding heat or pressure when using the assembly material 63 in the affixing. FIG. 11B is a schematic end view showing the distal portion of the guidewire 10 surrounded by the first tubular portion 15 with the assembly material 63 intervening therebetween. In other examples, the assembly material 63 intervenes therebetween in a portion of the circumference of the guidewire 10 and/or first tubular section 15 but not around the entire circumference.

In some embodiments, as illustrated schematically in FIG. 10B, the affixing step includes the use of an assembly tool 67. The assembly tool 67 can include, for example, but not exhaustively: a crimping tool or jig, a heat-shrinking tool, a welding or brazing tool, or a heat-welding tool. In embodiments, the assembly took 67 applies at least one of heat and pressure to secure the distal portion of the guidewire 10 within the first tubular section 15. FIG. 11C is a schematic end view showing the distal portion of the guidewire 10 surrounded by the first tubular portion 15, where at least a portion of the first tubular portion 15 has been mechanically and/or thermally deformed, e.g., by heat and/or pressure, so as to secure the distal portion of the guidewire 10 within.

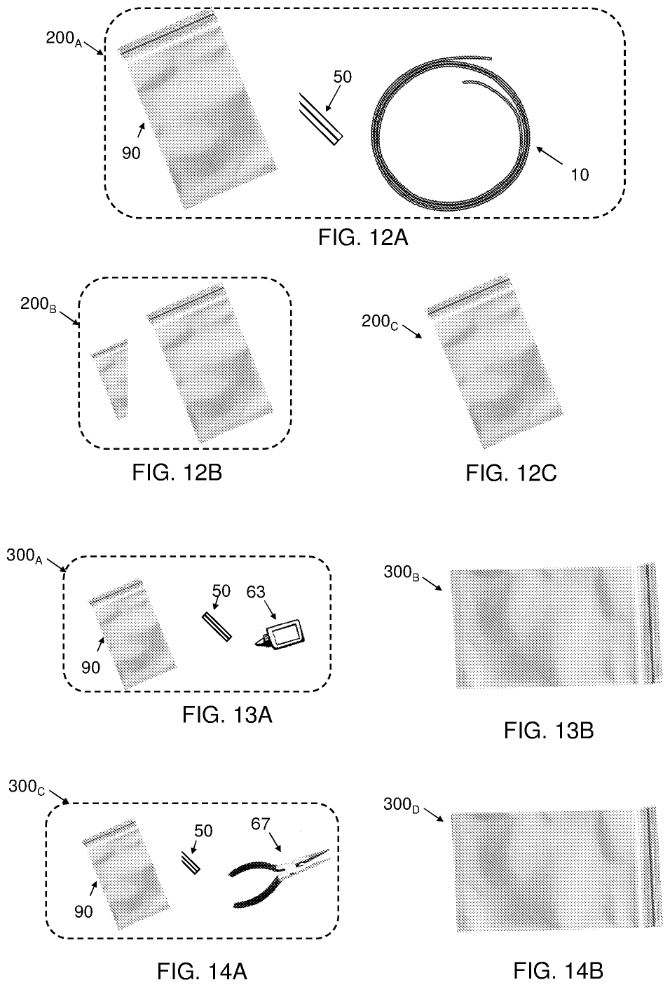

Reference is now made to FIGS. 12A-12C, 13A-13B, and 14A-14B. According to embodiments, the apparatus and devices disclosed herein can be provided in kits that can optionally include one or more containers such as sterile bags or pouches.

FIG. 12A shows a first kit in an unpackaged state, the first kit including (a) a coupling device 50 in accordance with any of the embodiments disclosed herein, (b) a guidewire 10, and, optionally, (c) an internally-sterile storage bag 90.

FIG. 12B shows the first kit arranged with the coupling device 50 packaged in a first storage bag 90, and the guidewire 10 packaged in a second storage bag 90.

FIG. 12C shows the first kit arranged with the coupling device 50 and the guidewire 10 packaged together in a single storage bag 90.

FIG. 13A shows a second kit in an unpackaged state, the second kit including (a) a coupling device 50 in accordance with any of the embodiments disclosed herein, (b) an assembly material 63, and, optionally, (c) an internally-sterile storage bag 90.

FIG. 13B shows a third kit, packaged together in a single storage bag 90, the third kit including (a) a coupling device 50 in accordance with any of the embodiments disclosed herein, (b) an assembly material 63, and a guidewire 10.

FIG. 14A shows a fourth kit in an unpackaged state, the fourth kit including (a) a coupling device 50 in accordance with any of the embodiments disclosed herein, (b) an assembly tool 67, and, optionally, (c) an internally-sterile storage bag 90.

FIG. 14B shows a fourth kit, packaged together in a single storage bag 90, the fourth kit including (a) a coupling device 50 in accordance with any of the embodiments disclosed herein, (b) an assembly tool 67, and a guidewire 10.

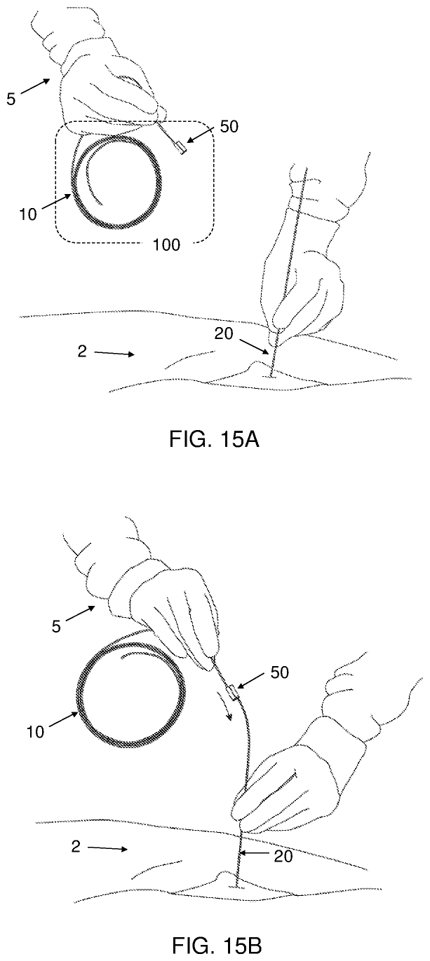

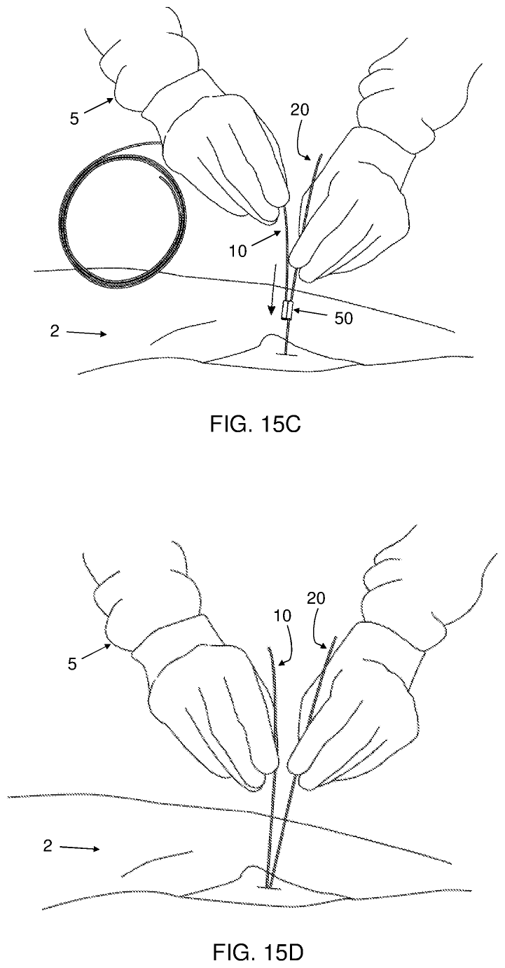

We now refer to FIGS. 15A-15D, which illustrate a method for coupling a first guidewire to a previously introduced second guidewire during a medical procedure.

In a first method step, an apparatus 100 comprising a coupling device 50 in accordance with any of the embodiments disclosed herein and a first guidewire 10 are provided. Assembly of the apparatus 100 can suitably be accomplished by using the method disclosed hereinabove in connection with the discussion of FIGS. 9-11C. The coupling device 50 comprises first and second tubular portions 15, 25 joined laterally in a fixed orientation to each other along the lengths of respective overlapping longitudinal sections. The lateral joining is such that respective longitudinal centerlines of the first and second tubular portions 15, 25 are parallel to each other or within ±10° of parallel in said fixed orientation. The coupling device 50 is affixed to the distal portion of the first guidewire 10 such that a longitudinal section of the first tubular portion 15 surrounds the distal portion of the first guidewire 15. FIG. 15A shows a surgeon 5 holding a proximal portion of a ‘second’ guidewire 20 previously introduced in a lumen of patient 2. The surgeon 5 also holds the provided apparatus 100 comprising, and a ‘first’ guidewire 10.

A second method step is illustrated in FIG. 15B, wherein the surgeon 5 slidably passes an end of the second guidewire 20 through the second tubular portion 25 of the coupling device 50 in a straight-line path between respective proximal and distal openings 27, 28 of the second tubular section 25. The straight-line path has a length-to-diameter ratio of at least 10:1.

FIGS. 15C and 15D illustrate additional, optional method steps. In FIG. 15C, the surgeon 5 is shown sliding the coupling device 50 distally along the second guidewire 20 in preparation for inserting the first guidewire 10 into the lumen or vessel of the patient 2. FIG. 15D shows the surgeon 5 holding both respective proximal ends of the guidewires 10, 20, both of which have been introduced into the lumen or vessel of the patient 2, the ‘first’ guidewire 10 introduced using the coupling device 50 and the associated methods disclosed in the embodiments.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. Apparatus comprising:
a. a first guidewire for use in a medical procedure; and
b. a coupling device comprising:
i. a first tubular portion formed to accommodate, through at least a proximal majority of a length thereof, passage of a distal portion of the first guidewire, and
ii. a second tubular portion formed to accommodate slidable passage therethrough of a second guidewire in a straight-line path between respective proximal and distal openings of the second tubular section, the straight-line path having a length-to-diameter ratio of at least 10:1,
the first and second tubular sections being joined laterally in a fixed orientation to each other along the lengths of respective overlapping longitudinal sections, the lateral joining being such that respective longitudinal centerlines of the first and second tubular portions are parallel to each other or within ±10° of parallel in said fixed orientation,
wherein the coupling device is affixed to the distal portion of the first guidewire such that a longitudinal section of the first tubular portion surrounds the distal portion of the first guidewire, and at least part of the longitudinal section of the first tubular portion that surrounds the distal portion of the first guidewire is deformed as a result of an affixing process.

2. The apparatus of claim 1, wherein the lateral joining is such that the respective longitudinal centerlines are parallel to each other or within ±5° of parallel, in said fixed orientation.

3. The apparatus of claim 1, wherein the lateral joining is such that a distance between respective distal ends of the first and second tubular sections is less than 2 cm.

4-5. (canceled)

6. The apparatus of claim 1, wherein the respective overlapping longitudinal sections have a length of at least 1 cm.

7-9. (canceled)

10. The apparatus of claim 1, wherein the length of the second tubular portion is at least 1 cm.

11-15. (canceled)

16. The apparatus of claim 1, additionally comprising an assembly material used in an affixing process.

17-22. (canceled)

23. A method of coupling a first guidewire to a previously introduced second guidewire during a medical procedure, the method comprising:
a. affixing a coupling device to a distal portion of the first guidewire, the coupling device comprising two first tubular portions joined laterally in a fixed orientation to each other along the lengths of respective overlapping longitudinal sections, the lateral joining being such that respective longitudinal centerlines of the first and second tubular portions are parallel to each other or within ±10° of parallel in said fixed orientation, the affixing being such that that a longitudinal section of a first tubular portion of the coupling device surrounds a distal portion of the first guidewire and the affixing includes deforming at least part of the longitudinal section of the first tubular portion that surrounds the distal portion of the first guidewire using an assembly tool; and
b. slidably passing an end of the second guidewire through a second tubular portion of the coupling device in a straight-line path between respective proximal and distal openings of the second tubular section, the straight-line path having a length-to-diameter ratio of at least 10:1.

24-37. (canceled)

38. The method of claim 23, wherein the affixing includes using an assembly material.

39-54. (canceled)

55. A method of assembling an apparatus, comprising:
a. providing a guidewire;
b. providing a coupling device comprising a first tubular portion, and a second tubular portion joined laterally in a fixed orientation to the first tubular portion along the lengths of respective overlapping longitudinal sections; and
c. affixing the coupling device to the distal portion of the guidewire, such that that a longitudinal section of a first tubular portion of the coupling device surrounds a distal portion of the guidewire, wherein the affixing includes deforming at least part of the longitudinal section of the first tubular portion that surrounds the distal portion of the first guidewire using an assembly tool,
wherein the second tubular portion is formed to accommodate slidable passage therethrough of a different guidewire in a straight-line path between respective proximal and distal openings of the second tubular section, the straight-line path having a length-to-diameter ratio of at least 10:1.

56. (canceled)

57. The method of claim 55, wherein the affixing includes using an assembly material.

58. A method of coupling a first guidewire to a previously introduced second guidewire during a medical procedure, the method comprising:
a. providing the apparatus of claim 1, the apparatus comprising the first guidewire, and
b. slidably passing an end of the second guidewire through a second tubular portion of the coupling device in a straight-line path between respective proximal and distal openings of the second tubular section, the straight-line path having a length-to-diameter ratio of at least 10:1.
